(19) United States
(12) Patent Application Publication
(10) Pub. No.: US 2023/0110195 A1
(43) Pub. Date: Apr. 13, 2023

(54) SYSTEM FOR IMPLANTATION OF A MEDICAL IMPLANT IN THE HUMAN OR ANIMAL BODY

(71) Applicant: PFM MEDICAL AG, Koeln (DE)

(72) Inventor: Joachim SCHAEFER, Nonnweiler (DE)

(21) Appl. No.: 17/907,009

(22) PCT Filed: Mar. 22, 2021
(86) PCT No.: PCT/EP2021/057223

§ 371 Date: Sep. 22, 2022

(30) Foreign Application Priority Data
Mar. 25, 2020 (IT) 102020000006286

(51) Int. Cl. A61B 17/221

(52) U.S. Cl. CPC A61B 17/221; A61B 2017/00867

(57) ABSTRACT

A system for implanting a medical implant in the human or animal body, comprising: a wire-like element for connecting the system to the medical implant, a double-lumen guide tube for the wire-like element, and a handle for handling the system, wherein the medical implant is arranged at the proximal end of the guide tube and the handle is arranged at the distal end of the guide tube, and wherein the wire-like element is fixed with a first end to the handle, is guided through a first lumen of the double-lumen guide tube to the medical implant, engages with a connecting device of the medical implant and is guided through a second lumen of the double-lumen guide tube to the handle and is detachably fixed to the handle with a second end.

### FIELD

The invention relates to a system for implanting a medical implant in the human or animal body. A medical implant is an artificial device implanted in the human or animal body, which remains permanently or temporarily within the human or animal body.

### BACKGROUND

An example of a medical implant is a so-called stent, which is inserted into hollow organs of the human or animal body to keep them open. A stent can be, for example, a small tubular lattice structure made of metal or synthetic fibers. Stents are used in blood vessels, especially in the coronary arteries, to prevent reocclusion after dilatation. On the other hand, stents are used in cancer treatment to keep constrictions caused by malignant tumors open after dilatation.

Furthermore, medical implants are used in cardiac surgery, where the medical implants are mostly used to close an abnormal opening within the heart. Examples of such applications are the treatment of ventricular septal defects (VSD), persistent foramen ovale (PFO), persistent ductus arteriosus (PDA), atrial septal defects (ASD) or left atrial appendage (LAA) closure.

As far as possible, such medical implants are inserted into the human or animal body by means of a minimally invasive procedure. In a minimally invasive procedure, access is gained to a large blood vessel within the human or animal body, through which a catheter is inserted into the human or animal body. Via the blood vessels, this catheter is guided to the implantation site. The medical implant is then transported via the catheter using an insertion aid.

Typically, the medical implants are made of a shape memory material so that the medical implant unfolds into a predetermined shape after exiting the catheter. Thus, the medical implant is transported via the catheter to the implantation site by means of the insertion aid and unfolds into the predetermined shape after exiting the catheter. After the medical implant has been correctly inserted into the human or animal body at the implantation site, the connection between the insertion aid and the medical implant can be released.

Consequently, the medical implant must be connected as tightly as possible to the insertion aid during transport through the catheter and at the same time be easily detached from the insertion aid after implantation has been completed. In the prior art, for example, it is known to clamp the medical implant onto a carrier wire of the insertion aid and to release the clamp connection after successful implantation of the medical implant. However, a disadvantage of such a connection between the medical implant and the insertion aid is that a relatively high force is required to release the connection between the implant and the carrier wire, and furthermore the connection between the medical implant and the carrier wire, which is based on a frictional force, can be negatively influenced by liquids, for example, so that the connection between the medical implant and the carrier wire can loosen prematurely.

WO 2015/078807 A1 discloses a system for connecting a medical implant to an insertion aid, wherein the medical implant and the insertion aid each have a spiral-shaped connection region which, in the connected state, engage with one another and are fixed to one another relative to one another by a locking wire. However, this requires a specially designed spiral-shaped connection area on the medical implant.

### SUMMARY

The invention is thus based on the task of providing a system for implanting a medical implant in the human or animal body, which ensures a secure connection between the medical implant and the insertion aid during implantation of the medical implant in the human or animal body and, at the same time, enables the connection between the medical implant and the insertion aid to be easily released after implantation of the medical implant has been completed. Furthermore, due to an application in minimally invasive surgery, the connection between the medical implant and the insertion aid should have a diameter that is as small as possible, preferably a diameter that is smaller than or equal to the diameter of the medical implant or the insertion aid.

According to the invention, the task is solved by a system for implanting a medical implant in the human or animal body, comprising:
a wire-like element for connecting the system to the medical implant,
a double-lumen guide tube for the wire-like element, and
a handle for handling the system,

wherein the medical implant is disposed at the proximal end of the guide tube and the handle is disposed at the distal end of the guide tube, and wherein the wire-like member is fixed to the handle by a first end, is passed through a first lumen of the double-lumen guide tube to the medical implant, engages a connecting means of the medical implant, and is passed through a second lumen of the double-lumen guide tube to the handle and is releasably fixed to the handle by a second end.

For example, during manufacture of the system according to the invention, the wire-like element is passed through a connecting device such as a loop or eyelet of the medical implant to form a releasable connection between the system according to the invention and the medical implant. A first end of the wire-like element is subsequently passed through the first lumen of the double-lumen guide tube, and the second end of the wire-like element is passed through the second lumen of the double-lumen guide tube. This fixes the medical implant to the proximal end of the double-lumen guide tube. At the distal end of the double-lumen guide tube, one end of the wire-like member is fixed to the handle, while the other end of the wire-like member is releasably connected to the handle. By means of the system thus produced, the medical implant can be implanted via a catheter already laid to the implantation site in the human or animal body. After the medical implant has been correctly positioned in the human body, which is verified by means of an imaging method, for example, the connection between the system according to the invention and the medical implant is released so that the medical implant remains in the human or animal body and the system according to the invention can be removed again. To do this, the releasable connection between the handle and one end of it wire-like element is released. By pulling on the handle, the wire-like element is pulled out of the human or animal body by fixing the other end of the wire-like element to the handle. This simultaneously pulls the wire-like member out of the loop or eyelet of the medical implant, thereby releasing the connection between the system of the invention and the medical implant. After this connection is released, the double-lumen tubing can be removed from the human or animal body.

The proximal end of the double-lumen guide tube in the sense of the invention is the end facing the human or animal body, and the distal end of the double-lumen guide tube in the sense of the invention is the end facing away from the human body.

The connection between the system according to the invention and the medical implant has a very small diameter, since only the wire-like element has to be guided through an eyelet or loop of the medical implant. No additional latch wire or the like is required, nor are there any specially shaped connecting sections on the medical implant and/or the insertion aid as disclosed in the prior art. Thus, the diameter of the catheter required for implantation can be reduced.

The double-lumen guide tube prevents the wire-like element from twisting into each other, which would make it much more difficult to remove and thus detach the medical implant. Only one section of the wire-like element is guided through each lumen of the double-lumen guide tube.

The system according to the invention comprises a very small number of components, which at the same time have as simple a structure as possible. As a result, the system according to the invention can be manufactured simply and inexpensively. Furthermore, storage, packaging and transport costs are reduced. Also, the handling is very simple and intuitive and the user does not need to have any special knowledge to use it.

According to an advantageous variant of the invention, the wire-like element is made of a metal, in particular stainless steel. A wire-like element made of metal has a high tensile strength with a small diameter, whereby the diameter of the system according to the invention can be minimized. This has a positive effect on implantation, in particular since a catheter with a smaller diameter can be used for implantation.

According to an expedient variant of the invention, the wire-like element has a diameter of 0.1 mm to 0.5 mm, in particular a diameter of 0.2 mm.

In a preferred embodiment of the invention, the double-lumen guide tube consists at least in part of a plastic. This makes the double-lumen guide tube flexible, which simplifies guiding the guide tube through the vascular system of the human or animal body. For example, a flexible guide tube adapts to the shape of a catheter that has already been placed. The mechanical properties of the double-lumen guide tube can further be adapted by reinforcing elements such as metal elements integrated in the plastic, in particular wires or metal grids. The guide tube can also be designed to be controllable by wires inserted into the plastic.

According to an expedient variant of the invention, the double-lumen guide tube has a diameter of 1.0 mm to 2.0 mm, in particular a diameter of 1.76 mm.

The individual lumens of the double-lumen guide tube have an inner diameter adapted to the diameter of the wire-like element, so that the wire-like element can be guided through the lumens of the double-lumen guide tube. In particular, the diameter of the lumens is at least slightly larger than the diameter of the wire-like element.

According to a variant of the invention, the double-lumen guide tube comprises two inner tube-like elements forming the first lumen and the second lumen of the double-lumen guide tube. Thus, the double-lumen guide tube comprises a total of three tube-like members, with two tube-like members disposed within an outer tube-like member. The two inner tube-like elements thereby form the first lumen and the second lumen of the double-lumen guide tube.

The invention is also not limited to a double lumen guide tube, but can also be realized with a multi-lumen tube. However, only two lumens are required for guiding the wire-like element. Any additional lumens may be used for other purposes, such as injection of fluids such as contrast agents for imaging procedures or injection or insertion of other fluids, objects and/or devices.

In an expedient variant of the invention, the two internal hose-like elements are each designed as a stainless steel helix. Such stainless steel helixes exhibit high radial flexibility with simultaneous stability in the longitudinal direction.

According to an advantageous variant of the invention, the connecting device of the medical implant is formed as an eyelet or loop. Such an eyelet or loop can be easily formed on the medical implant, since medical implants are often made of wire-like elements. It is necessary to form an eyelet or loop as only from the wire-like element of the medical implant, for example at one end of the medical implant.

According to a further variant of the invention, the first end of the wire-like element comprises a nipple which can be fixed in a corresponding recess of the handle. This allows the first end of the wire-like element to be easily fixed to the handle. However, other fixations are also possible, such as gluing, welding, soldering and the like.

In a preferred embodiment of the invention, the handle comprises a clamping mechanism for the second end of the wire-like member, for releasably fixing the second end of the wire-like member to the handle. The clamping mechanism allows the second end to be releasably fixed to the handle. After successful implantation of the medical implant, the clamping connection is released and the connection between the system according to the invention and the medical implant can be released by pulling the wire-like element out of the eyelet or loop of the medical implant. This is achieved by fixing the first end of the wire-like element to the handle and disconnecting the second end of the wire-like element and pulling the wire-like element by means of the handle.

According to an advantageous variant of the invention, the clamping mechanism comprises a safeguard against unintentional actuation of the clamping mechanism, in particular against unintentional release of the clamping mechanism. The safety device must therefore be released before the clamping mechanism is actuated.

According to another useful variant of the invention, the system according to the invention comprises an implantation catheter for guiding the double-lumen guide tube and the medical implant connected thereto to the implantation site in the human or animal body. The implantation catheter is guided through the vascular system of the human or animal body to the implantation site. For this purpose, for example, access is made to a major vessel of the human or animal body, in particular in the groin region of the human body. A steerable catheter may be directed through the vascular system to the implantation site in the human or animal body. Alternatively, the implantation catheter is guided to the implantation site by means of a guide wire that has already been placed. Via the implantation catheter, the medical implant is advanced through the implantation catheter by means of the double-lumen guide tube. The double-lumen guide tube assumes the function of a so-called pusher, which advances the medical implant through the implantation catheter to the implantation site. After successful implantation, the connection of the system according to the invention to the medical implant is released as described above and the double-lumen guide tube and the implantation catheter can be removed from the human or animal body.

In one embodiment of the invention, the system additionally comprises a Y-connector for flushing the system, in particular the double-lumen guide tube and/or the implantation catheter.

According to another embodiment, the system further comprises a so-called pusher to move the double-lumen guide tube and the medical implant connected thereto through the implantation catheter.

### BRIEF DESCRIPTION OF THE DRAWINGS

The invention is explained in more detail below with reference to the embodiments shown in the figures. They show:

FIG. 1 is a view of a system according to the invention;

FIG. 2 is a detailed view of the proximal end of the system from FIG. 1;

FIG. 3 is a detailed view of a handle of a system according to the invention;

FIG. 4 is a detailed view of a medical implant connected to a system according to the invention; and

FIG. 5 is a cross-sectional view of a double-lumen guide tube of a system according to the invention.

### DETAILED DESCRIPTION

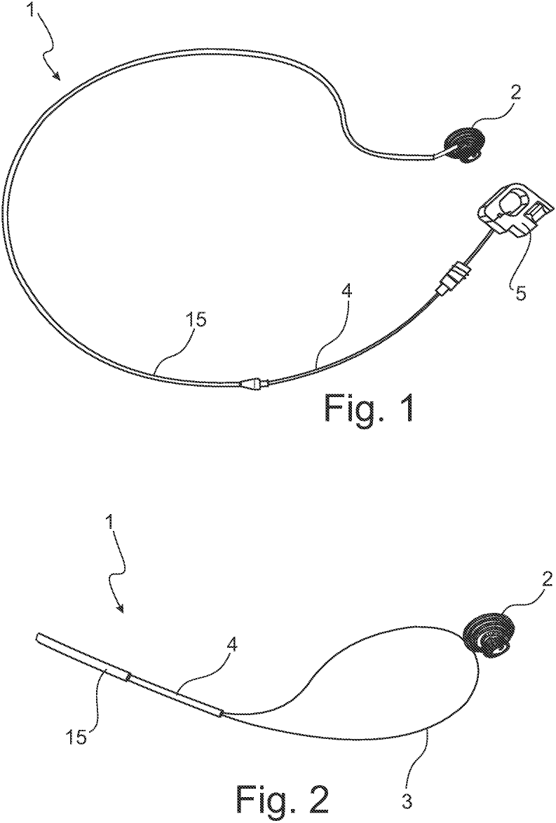

FIG. 1 shows an exemplary embodiment of a system 1 according to the invention for implanting a medical implant 2 in the human or animal body. The system 1 comprises a wire-like element 3 for connecting the system 1 to the medical implant 2. The system 1 further comprises a double-lumen guide tube 4 for the wire-like element 3 and a handle 5 for handling the system 1.

The medical implant 2 is arranged at the proximal end of the guide tube 4 and the handle 5 is arranged at the distal end of the guide tube 4, wherein proximal concerns the end of the guide tube 4 facing the human or animal body and distal concerns the end of the guide tube 4 facing away from the human or animal body.

The wire-like element 3 is fixed with a first end 9 to the handle 5, is guided through a first lumen 6 of the double-lumen guide tube 4 to the medical implant 2, subsequently engages with a connecting device 8 of the medical implant 2 and is then guided through a second lumen 7 of the double-lumen guide tube 4 to the handle 5. At the handle 5, the second end 10 of the wire-like element 3 is detachably fixed.

According to the embodiment example of FIG. 1, the system 1 according to the invention further comprises an implantation catheter 15, by means of which the medical implant 2 is advanced to the implantation site in the human or animal body by means of the double-lumen guide tube 4.

The wire-like element 3 consists, for example, of a metal, in particular stainless steel, and has a diameter of 0.1 mm to 0.5 mm, in particular 0.2 mm.

The double-lumen guide tube 4 consists, for example, at least partially of a plastic and has a diameter of 1.0 mm to 2.0 mm, in particular 1.76 mm.

The connecting device 8 of the medical implant 2 is preferably designed as an eyelet or loop.

A so-called pusher is arranged between the implantation catheter 15 and the handle 5 in order to move the double-lumen guide tube 4 and the medical implant 2 fixed therein through the implantation catheter 15.

Furthermore, a Y-connector can be arranged at the transition between implantation catheter 15 and double-lumen guide tube 4, for flushing the system 1 according to the invention, in particular the implantation catheter 15.

FIG. 2 shows a detailed view of the connection of the system 1 according to the invention with a medical implant 2 at the proximal end of the system 1. The double-lumen guide tube 4 with the medical implant 2 attached to it emerges from the implantation catheter 15. The medical implant 2 is connected to the system 1 according to the invention by the wire-like element 3, wherein the wire-like element 2 engages a connecting means 8 of the medical implant. The connecting device 8 of the medical implant is formed, for example, as an eyelet or loop. On their way to the distal end of the system 1 according to the invention, the two ends 9, 10 of the wire-like element are guided through the first lumen 6 and the second lumen 7 of the double-lumen guide tube 4.

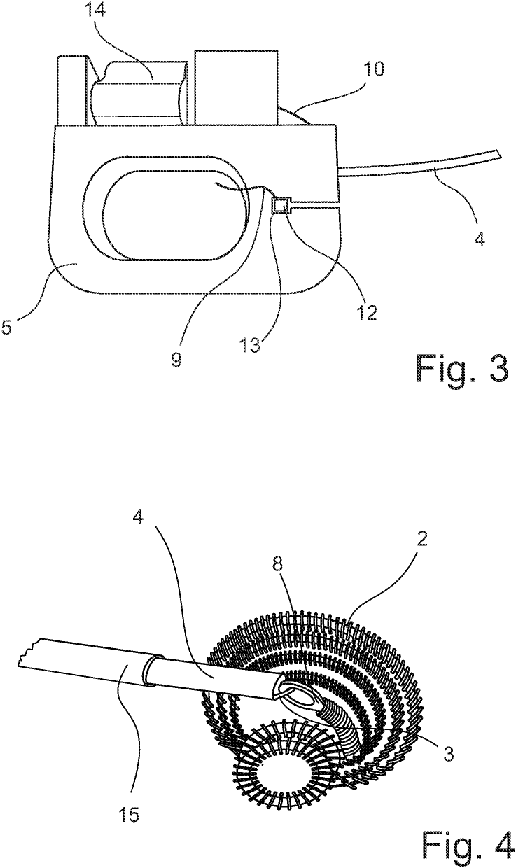

FIG. 3 is a detailed view of a handle 5 of a system 1 according to the invention for implanting a medical implant 2 in the human or animal body. A first end 9 of the wire-like element 4 is fixedly attached to the handle 5. For this purpose, the wire-like 4 has, for example, a nipple 12 at the first end, which engages in a corresponding recess 13 on the handle 5. However, other fixations are also possible, such as gluing, welding, soldering or other connection techniques. The second end 10 of the wire-like element 4 is detachably connected to the handle 5, for example by means of a clamping mechanism 14.

After a successful implantation of the medical implant 2, the connection between second end 10 of the wire-like element 4 and the handle 5 is released and the wire-like element 4 is pulled out of the connection device 8 of the medical implant 2 to release the connection between the system 1 according to the invention and the medical implant 2.

According to an expedient variant of the invention, the clamping mechanism 14 can comprise a safety device (not shown) which prevents unintentional actuation of the clamping mechanism 14. The safety device is designed, for example, as a latch.

FIG. 4 shows a detailed view of a medical implant 2 connected to a system 1 according to the invention. The double-lumen guide tube 4 emerges from the implantation catheter 15. Through the first lumen 6 of the double-lumen guide tube 4, the wire-like element 3 is guided from the handle 5 of the system 1 according to the invention to the medical implant 2. The wire-like element 3 engages the connection device 8 of the medical implant 2 and is guided back to the handle 5 of the system 1 according to the invention through the second lumen 7. A first end 9 of the wire-like element 3 is fixed to the handle 5, while the second end 10 is detachably fixed to the handle 5. To eject the medical implant 2 from the system 1 according to the invention, the releasable connection between the handle 5 and the second end 10 of the wire-like element 3 is released and the wire-like element is pulled out of the double-lumen guide tube 4 by means of the handle 5 and the first end 9 fixed thereto, until the wire-like element 3 no longer engages the connection device 8 of the medical implant 2. Subsequently, the double-lumen guide tube 4 can be pulled out of the implantation catheter 15 and finally the implantation catheter 15 is removed from the human or animal body.

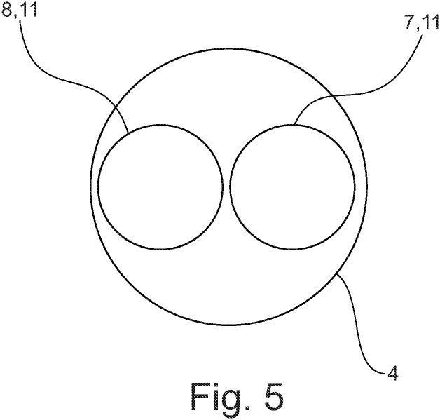

FIG. 5 shows a cross-sectional view of a double-lumen guide tube 4 of a system 1 according to the invention. According to the embodiment example shown in FIG. 5, the double-lumen guide tube 4 comprises two internal tube-like elements 11, which form the first lumen 6 and the second lumen 7 of the double-lumen guide tube 4. The two inner tube-like elements 11 are, for example, each formed as a stainless steel helix.

List of reference signs

| | |
|---|---|
| 1 | System |
| 2 | medical implant |
| 3 | wire type element |
| 4 | double lumen guide hose |
| 5 | handle |
| 6 | first lumen |
| 7 | second lumen |
| 8 | connection device (medical implant) |
| 9 | first end (wire-like element) |
| 10 | second end (wire-like element) |
| 11 | inside tubular elements |
| 12 | nipples |
| 13 | recess |
| 14 | clamping mechanism |
| 15 | implantation catheter |

What is claimed is:

1-12. (canceled)

13. A system for implanting a medical implant in the human or animal body, comprising:
a wire-like element for connecting the system to the medical implant;
a double-lumen guide tube for the wire-like element; and
a handle for handling the system;
wherein the medical implant is arranged at the proximal end of the guide tube and the handle is arranged at the distal end of the guide tube, and wherein the wire-like element is fixed with a first end to the handle, is guided through a first lumen of the double-lumen guide tube to the medical implant, engages with a connecting device of the medical implant and is guided through a second lumen of the double-lumen guide tube to the handle and is detachably fixed to the handle with a second end.

14. The system according to claim 13, wherein the wire-like element is made of a metal, in particular stainless steel.

15. The system according to claim 13, wherein the wire-like element has a diameter of 0.1 mm to 0.5 mm, in particular 0.2 mm.

16. The system according to claim 13, wherein the double-lumen guide tube is at least partially made of a plastic.

17. The system according to claim 13, wherein the double-lumen guide tube has a diameter of 1.0 mm to 2.0 mm, in particular 1.76 mm.

18. The system according to claim 13, wherein the double-lumen guide tube comprises two internal tube-like elements forming the first lumen and the second lumen of the double-lumen guide tube.

19. The system according to claim 18, wherein the two internal hose-like elements are each formed as a stainless steel helix.

20. The system according to claim 13, wherein the connecting device of the medical implant is formed as an eyelet or loop.

21. The system according to claim 13, wherein the first end of the wire-like element comprises a nipple fixable in a corresponding recess of the handle.

22. The system according to claim 13, wherein the handle comprises a clamping mechanism for the second end of the wire-like element, for releasably fixing the second end of the wire-like element to the handle.

23. The system according to claim 22, wherein the clamping mechanism comprises a safeguard against unintentional actuation of the clamping mechanism.

24. The system according to claim 13, further comprising an implantation catheter for guiding the double-lumen guide tube and the medical implant connected thereto to the implantation site in the human or animal body.
